(19) United States
(12) Patent Application Publication
(10) Pub. No.: US 2021/0204685 A1
(43) Pub. Date: Jul. 8, 2021

(54) FOLDING SELF-CLEANING TOOTHBRUSH

(71) Applicant: JEKLO LLC, Orem, UT (US)

(72) Inventors: Jacob Christensen, Murray, UT (US); Travis Lish, Orem, UT (US)

(21) Appl. No.: 17/138,556

(22) Filed: Dec. 30, 2020

Related U.S. Application Data
(60) Provisional application No. 62/956,873, filed on Jan. 3, 2020.

(51) Int. Cl. A46B 17/06; A61C 17/22; A46B 5/00; A46B 15/00; A61L 2/10; A61L 2/24

(52) U.S. Cl. CPC A46B 17/065; A61C 17/225; A46B 5/0058; A46B 5/0095; A46B 15/001; A61L 2202/11; A46B 15/0095; A61L 2/10; A61L 2/24; A46B 2200/1066; A46B 15/0038

(57) ABSTRACT

A folding self-cleaning toothbrush is configured with of a head having bristles, a handle with an open recess wherein the head can be stored, a joint which allows the head to fold into the handle, a battery, and a sanitization device with LED lights that emit light in a spectrum suitable for cleaning the head/bristles. The head may be detachable and disposable. The toothbrush also has a computing system for controlling activation of the UV light emitter/sanitization device, as well as to generate notifications related to use and/or cleaning of the toothbrush.

### CROSS-REFERENCE TO RELATED APPLICATIONS

This application claims the benefit of and priority to U.S. Provisional Patent Application Ser. No. 62/956,873 filed on Jan. 3, 2020, entitled “FOLDING SELF-CLEANING TOOTHBRUSH,” which is incorporated herein by reference in its entirety.

### BACKGROUND

### Technical Field

This disclosure generally relates to dental care instruments. More specifically, the present disclosure relates to self-cleaning dental care instruments.

### Related Technology

Over 40% of adults that are 30 years old or older have gum disease. Similarly, tooth decay is common among adults and children. These dental diseases can cause severe pain and discomfort, and thus, greatly reduce a person's quality of life. However, good oral hygiene can prevent a large percent of dental diseases.

Good oral hygiene includes tooth brushing, tongue scraping and flossing. Using mouthwash and visiting the dentist regularly can also improve oral health. However, twice-a-day tooth brushing is the most important and ubiquitous form of oral care.

Toothbrushes have remained largely unchanged for over a thousand years. For example, bristle toothbrushes have been found in China and date back to circa 800 AD. Despite the toothbrush's long and prestigious history, the dental hygiene tool has some shortcomings.

For example, toothbrushes are often stored within or near a bathroom facility. Toothbrushes are also commonly stored next to other toothbrushes. Because of this proximity, a toothbrush is exposed to many potentially harmful germs through both airborne particulates and through direct contact with bathroom surfaces. Additionally, a toothbrush provides an ideal environment (i.e., a warm and moist environment) for bacteria to grow and thrive.

Users also have difficulties while traveling with their toothbrushes. In many cases, a traveler will simply throw their unprotected toothbrush into a bag or suitcase. Thus, the toothbrush is introduced to bacteria and viruses from a user's clothes, shoes, electronics and other goods. Other users attempt to protect their toothbrush by putting it inside a protective case (e.g., a sealable sandwich bag). However, the makeshift cases often trap in moisture and germs, and thus expedite the spread and growth of bacteria on the toothbrush and the case.

Accordingly, there are several disadvantages with toothbrushes that can be addressed.

### BRIEF SUMMARY

Disclosed embodiments are directed to toothbrushes and, more particularly, to folding and self-cleaning toothbrushes.

In some embodiments, a folding self-cleaning toothbrush comprises a head with bristles, a handle forming a recess that is sized and shaped to receive at least the bristles of the head, a joint between the head and the handle about which the head can rotate from a first position with the bristles outside of the handle to a second position with the head folded towards the handle and in which the bristles are contained inside of the recess of the handle, a battery and a sanitization device that is operably powered by the battery for emitting a sanitizing light into the recess of the handle when the head is in the second position, the light comprising a spectrum of light that is operable to disinfect the bristles. Additionally, in certain embodiments, the head of the toothbrush is detachable from the handle.

In some embodiments the folding toothbrush is configured with a computing system comprising a processor that executes stored computer-executable instructions for controlling activation of the UV light emitter/sanitization device, as well as to generate notifications, such as a notification for replacing the head and/or for letting the user know when a sanitization process is complete or a battery is running low and/or for controlling a motor to oscillate, rotate or otherwise vibrate the head/bristles.

In some embodiments, the sanitization light consists of a UV-C light. Additionally, in certain embodiments, the sanitization device uses UV-C LEDs to emit the sanitizing light. In some instances, the sanitization device is configured to automatically emit the sanitizing light for a predetermined period when the toothbrush is folded into the second position. Additionally, in some instances, the folding toothbrush's computing system may analyze user data to intelligently and automatically set a time for the sanitization device to start a cycle based on tracked usage and/or user behaviors.

In some embodiments, a locking mechanism is provided for locking the head into the second position and a button which is operable, when pressed, to unlock the locking mechanism for removing the head from the second position and into the first position. The button may also automatically fold or unfold the toothbrush. In other words, the joint of the toothbrush may be spring-loaded with a spring, such that the button, when pressed with the head in the first position, unlocks the locking mechanism which enables the head to be automatically biased by the spring to move from the second position to the first position.

In certain embodiments, the folding toothbrush has a rechargeable battery and circuitry for recharging the battery when positioned on a recharging dock, such as through induction. Additionally, the folding toothbrush may include a Bluetooth transmitter and/or transceiver for communicating with one or more wireless systems and to receive control instructions and/or to transmit state and use data.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims or may be learned by the practice of the disclosure as set forth hereinafter.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which:

FIG. 1 illustrates a perspective view of a folding self-cleaning toothbrush according to one or more embodiments of the present disclosure.

FIGS. 2A and 2B illustrate a front view and back view respectively of a folding self-cleaning toothbrush according to one or more embodiments of the present disclosure.

FIGS. 3A and 3B illustrate a left view and right view respectively of a folding self-cleaning toothbrush according to one or more embodiments of the present disclosure.

FIG. 4 illustrates a perspective view of a folding self-cleaning toothbrush with a detached head according to one or more embodiments of the present disclosure.

FIGS. 5A and 5B illustrate a front view and back view respectively of a folding self-cleaning toothbrush with a detached head according to one or more embodiments of the present disclosure.

FIG. 6 illustrates a left view of a folding self-cleaning toothbrush in five different positions according to one or more embodiments of the present disclosure.

FIG. 7A illustrates a detailed and partial cross-section view of the locking mechanism of the self-cleaning toothbrush when the locking mechanism is engaged according to one or more embodiments of the present disclosure.

FIG. 7B illustrates a detailed and partial cross-section view of the locking mechanism of the self-cleaning toothbrush when the locking mechanism is not engaged according to one or more embodiments of the present disclosure.

FIG. 8 illustrates a perspective view of a folding self-cleaning toothbrush in the folded position according to one or more embodiments of the present disclosure.

FIGS. 9A and 9B illustrate a front view and back view respectively of a folding self-cleaning toothbrush in the folded position according to one or more embodiments of the present disclosure.

FIG. 10 illustrates an example computing system and circuitry that facilitate the operation of the folding self-cleaning toothbrush.

FIGS. 11A and 11B illustrate a bottom view and top view respectively of a folding self-cleaning toothbrush according to one or more embodiments of the present disclosure.

FIG. 12 illustrates a right view, partly in section, of a folding self-cleaning toothbrush according to one or more embodiments of the present disclosure.

FIG. 13 illustrates a front view of a folding self-cleaning toothbrush and a dock according to one or more embodiments of the present disclosure.

FIG. 14 illustrates a right view of a folding self-cleaning toothbrush and a dock according to one or more embodiments of the present disclosure.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

Furthermore, it is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

In addition, unless otherwise indicated, numbers expressing quantities, constituents, distances, or other measurements used in the specification and claims are to be understood as being modified by the term “about,” as that term is defined herein. Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Any headings and subheadings used herein are for organizational purposes only and are not meant to be used to limit the scope of the description or the claims.

### Folding Self-Cleaning Toothbrush Overview

The disclosed embodiments are generally directed to a folding self-cleaning toothbrush. In some embodiments, the toothbrush has a detachable head that is disposable and a handle with a sanitization device for cleaning the head. In some embodiments, the sanitization device comprises of 1 to 100 Ultraviolet (UV) LEDs which turn on automatically when the toothbrush is folded. For instance, in some embodiments, the folding of the head into the handle triggers a switch that is activated to power the LEDs for a predetermined period of time, as controlled by a processor in the handle. The switch, not shown, may be positioned in the joint or any part of the handle, which is mechanically pressed (as a control button), for activating the LED lighting function.

The handle also has a head storage cavity where the head can be stored. In other words, the handle has a recess that is sized and shaped to receive at least the bristles of the head. Additionally, the toothbrush has a joint that allows the toothbrush head to fold or rotate into the handle (i.e., the toothbrush head rotates into the head storage cavity). In other words, the toothbrush has a joint between the head and the handle about which the head can rotate from the first position (i.e., unfolded position) with the bristles outside of the handle to a second position (i.e., folded position) with the head folded towards the handle and in which the bristles are contained inside of the recess of the handle.

The handle also contains a battery to power the sanitization device and a computing system to control the electronic self-cleaning toothbrush. In some embodiments, the toothbrush can communicate wirelessly (e.g., through Wi-Fi or Bluetooth) with a user's cellphone or computer. The toothbrush system may also include a dock that automatically charges the folding toothbrush.

Some embodiments of the folding self-cleaning toothbrush are made of a lightweight material such as plastic, aluminum or titanium. The toothbrush can also be made of materials with antimicrobial properties (e.g., copper, brass). Furthermore, the folding toothbrush may have an antimicrobial coating. Additionally, some embodiments of the folding self-cleaning toothbrush are dust resistant, drop resistant and water resistant (e.g., IP68 rating).

Some embodiments of the folding toothbrush come with a fabric carrying case. The carrying case can be made of a breathable material (e.g., polyester, nylon) to reduce moisture levels of the toothbrush while further protecting the toothbrush from germs, dust and debris.

The disclosed embodiments overcome many of the disadvantages and limitations that are common to toothbrushes. For example, the sanitization device can conveniently eradicate a large percentage of bacteria and viruses on the toothbrush. Therefore, a user will not have to replace his or her toothbrush as often.

When the toothbrush does have to be replace, the detachable head allows a user to replace the head of the toothbrush without having to replace the entire toothbrush. Thus, the folding toothbrush allows a person to replace their toothbrush in a more cost-effective and eco-friendly manner.

The folding capabilities of the toothbrush also allows users to store the toothbrush in a convenient and sanitary manner. For example, a traveler can throw the folded toothbrush in his bags without worrying about it getting infected. Additionally, in some embodiments, the folding self-cleaning toothbrush has vents to reduce the buildup of moisture when in the folded position. Therefore, the toothbrush is protected from germs, moisture, and impact damage while in the folded position.

In some embodiments, the folded toothbrush is about half the size of a regular toothbrush. The toothbrush's size is also convenient for carrying in a purse, backpack or pocket. Thus, a person can easily take the toothbrush with them when on the go.

Overall, the folding self-cleaning toothbrush can be used to overcome many of the limitations of toothbrushes and oral hygiene instruments generally.

### Detachable Head

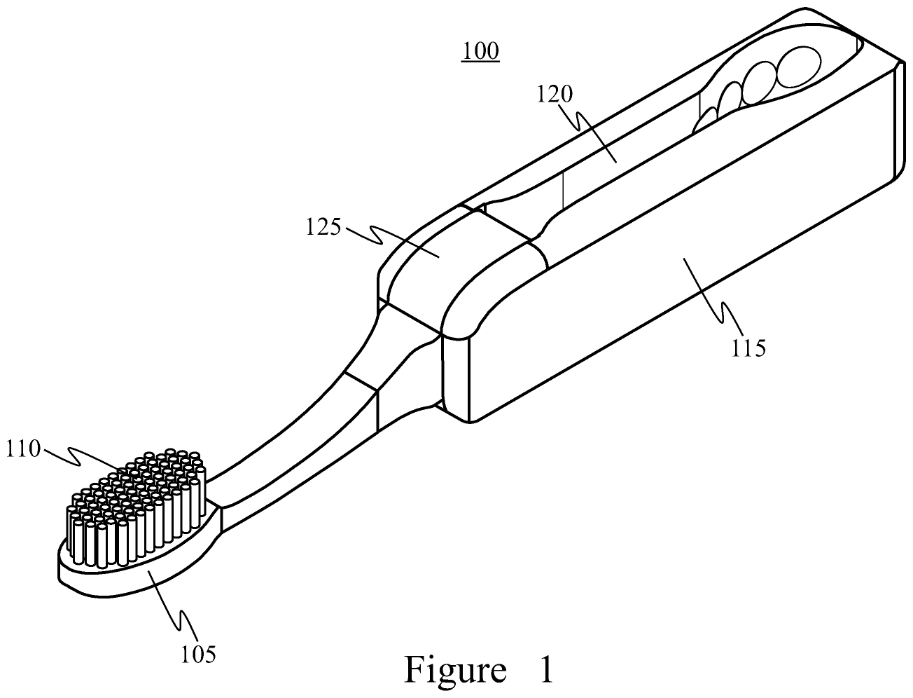

FIG. 1 illustrates a perspective view of one embodiment of the folding self-cleaning toothbrush 100. The toothbrush 100 has a head 105 that is detachable from the handle, a joint 125, and a handle 115.

In some embodiments, the height (i.e., from the top of the head 105 to the bottom of the handle 115) of the toothbrush 100 may vary to accommodate different needs and preferences, from 15 cm to 16 cm, 17 cm, 18 cm, 19 cm, 20 cm, 21 cm, 22 cm or more than 22 cm. In other embodiments, the height of the unfolded toothbrush 100 is much shorter (e.g., from 7 cm to 14 cm) to facilitate storage during travel. This travel version (not shown) of the toothbrush 100 is designed to fit comfortably inside a user's pocket.

In some embodiments, the detachable head 105 has bristles 110 on the top-front portion of the head. Additionally, the stiffness of the bristles 110 may vary to accommodate different needs and preferences. For example, users with sensitive teeth may need softer bristles. Additionally, the bristles can be made of UV resistant fibers so that the sanitization device does not damage the bristles 110.

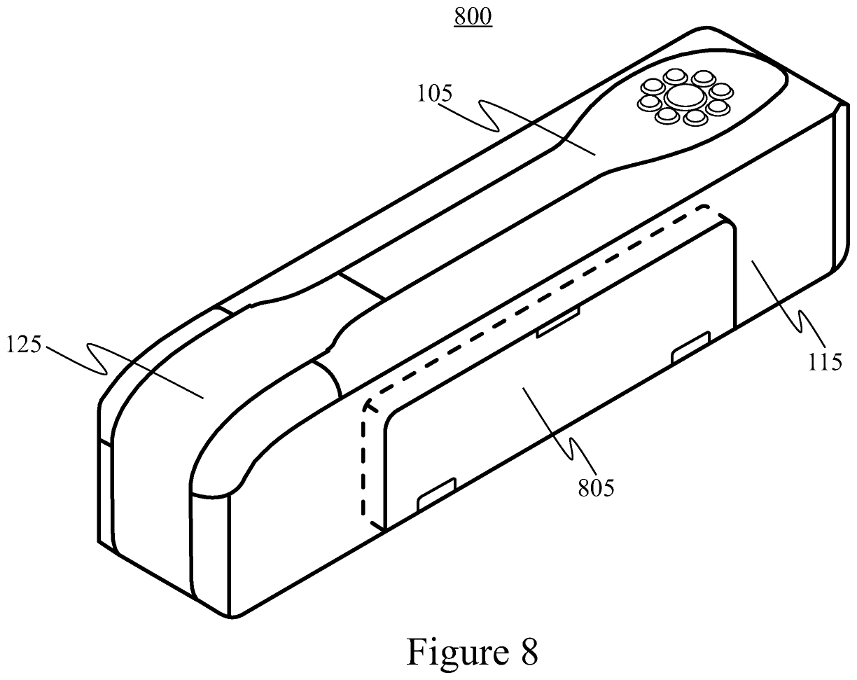

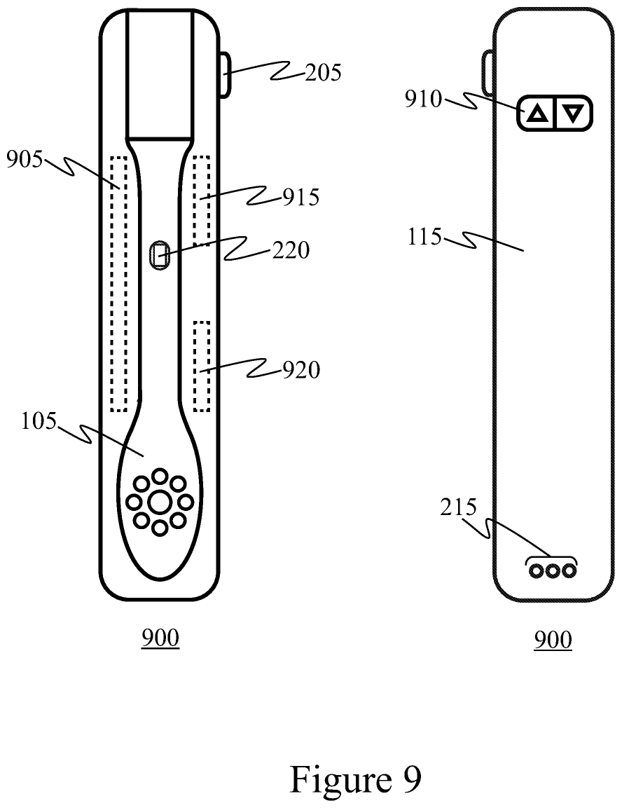

FIG. 1 also illustrates the head storage cavity 120. When the toothbrush 100 is in the second position (i.e., the folded position, see FIGS. 8 and 9), the detachable head 105 fits into the head storage cavity 120. The head storage cavity 120 is configured to be similar to the size and shape of the head 105 to ensure a close fit between the head 105 and the head storage cavity 120. In other words, the handle 115 forms a recess that is sized and shaped to receive at least the bristles 110 of the head 105. Additionally, while the head 105 is in the head storage cavity 120, the bristles 110 are protected from dust and germs.

In some embodiments, the head 105 can be replaced after a predetermined amount of time (e.g., 4 months) or after the bristles 110 have worn out. The head 105 may also be replaced if it is damaged by an impact (e.g., user drops the toothbrush). Additionally, in some embodiments, the detachable head 105 attaches to the top end of the joint 125. Thus, a user can replace the detachable head 105 and continue using the same handle 115 and joint 125. However, in some embodiments, the head is permanently attached and cannot be removed.

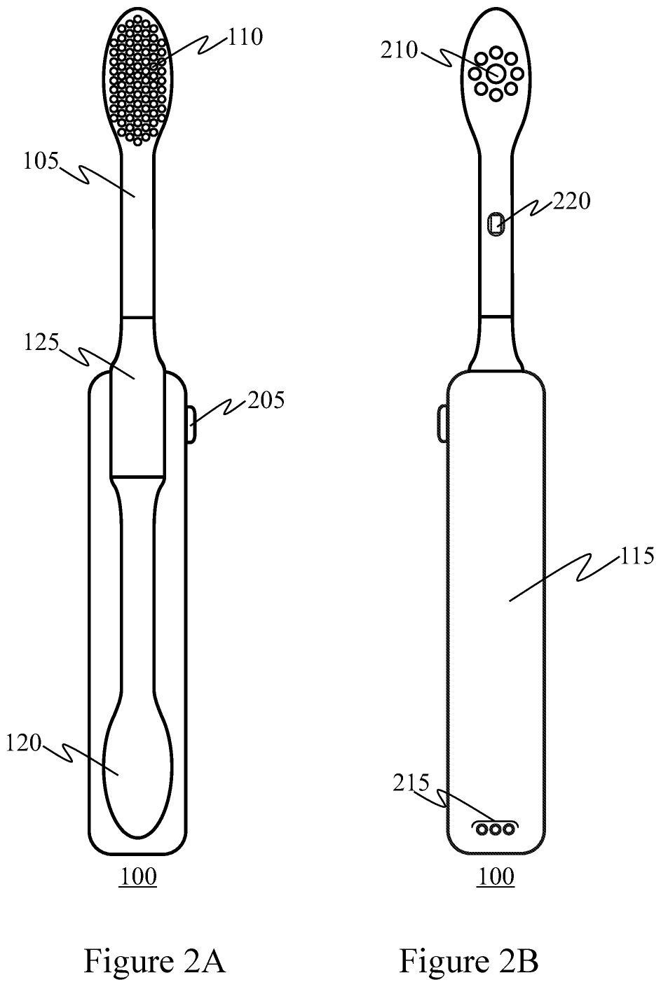

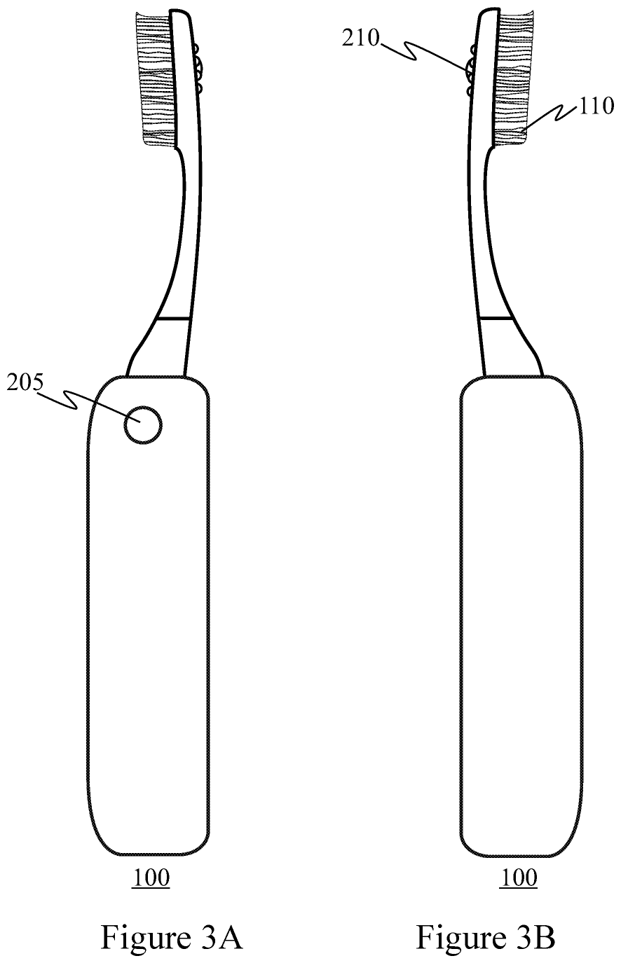

FIG. 2 illustrates a front and back view of the folding self-cleaning toothbrush 100 where the head 105 is attached. Similarly, FIG. 3 illustrates a left view and right view of the folding self-cleaning toothbrush 100. In some embodiments, the detachable head 105 can have one or more indicator LEDs 220 that alert the user when it is time to replace the head 105. For example, a red LED 220 can turn on when the head 105 has been used for more than the recommended period (e.g., three to six months). The indicator LED 220 can also indicate that the head 105 should be replaced soon (e.g., in less than two weeks). Thus, the user can ensure that he or she is replacing the head 105 at appropriate intervals in order to maintain the best possible oral health.

In some embodiments, the indicator LED 220 can also help users who have multiple toothbrushes 100 identify which toothbrush 100 is theirs. For example, a user can set their toothbrush 100 to glow a certain color when the toothbrush 100 is unfolded. Thus, if the user picks up the wrong toothbrush, he or she will notice that the indicator LED 220 color does not match his or her preset color. Additionally, in some embodiments, the indicator LED 220 is connected to the toothbrush's computing system and battery. Thus, the indicator LED 220 is powered by the battery and can receive and send data from the computing system.

In some embodiments, the toothbrush 100 further improves a user's oral health by providing them with a tongue cleaner 210. The tongue cleaner 210 consists of raised bumps on the back of the head 105 that can be used to scrape germs and dead cells off a user's tongue. Therefore, users can conveniently clean their tongue after brushing their teeth by simply flipping the toothbrush 100 over.

The folding self-cleaning toothbrush 100 may also include a button 205 that folds and/or unfolds the toothbrush 100. Additionally, in some embodiments, the toothbrush 100 has a charging port and/or charging contacts 215. Thus, the toothbrush can be recharged without removing the battery. More details about the button 205, charging contacts 215 and battery will be provided later.

In some embodiments, the head 105 has a vibrating motor (not shown) to help the user remove more plaque from his or her teeth and gums. Additionally, the vibrating motor helps users with limited mobility (e.g., because of carpal tunnel or arthritis) to brush their teeth more easily. The motor causes the bristles 110 to vibrate back and forth when the user activates the motor. In other words, the motor vibrates the bristles 110 on the head 105. The vibrating motor can be housed in either the head 105 or in the handle 115.

In some embodiments, the bristles 110 are attached to a rotating motor (not shown) which is housed in the head 105 or in the handle 115. Like the vibrating motor, the rotating motor can help users clean their teeth better and assists users with limited mobility. The control switch for the motor can be placed on either the handle 115 or directly on the head 105. Additionally, in some embodiments, the motor is connected to the toothbrush's computing system and battery. Thus, the motor is powered by the battery and can receive and send data from the computing system.

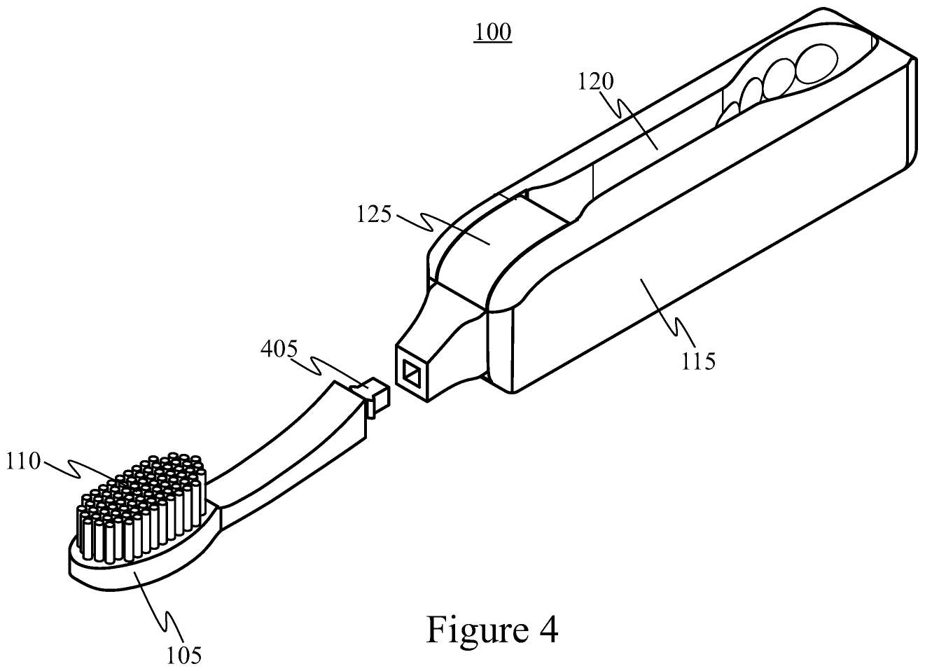

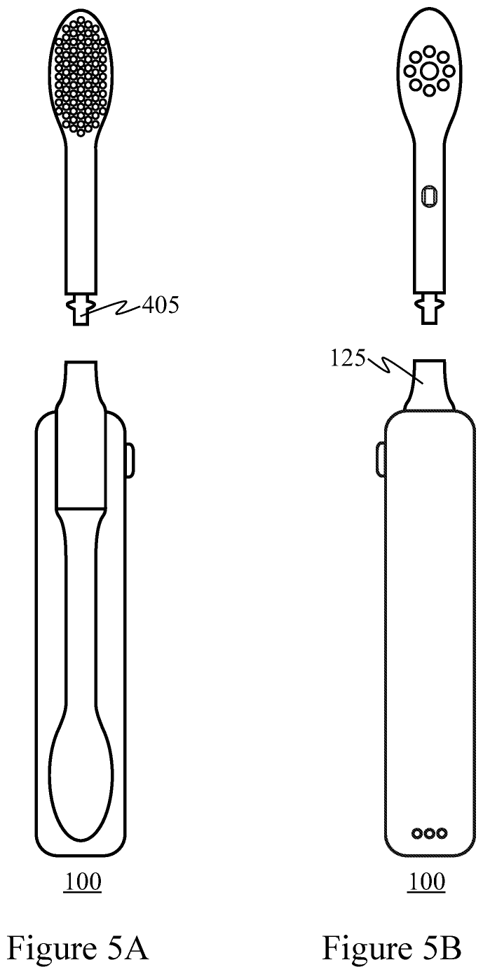

FIGS. 4 and 5 illustrate a perspective view, a front view and a back view of the folding self-cleaning toothbrush 100 where the head 105 has been detached. In some embodiments, the head 105 attaches to the joint 125 using a snap fit. In other words, a small protrusion on the attachment stem 405 deflects during assembly and catches on a lip inside the joint 125. Thus, a user can attach the head 105 by firmly pressing the attachment stem 405 into the joint 125. Similarly, a user can detach the head 105 by firmly pulling on the head 105 while holding the handle 115 in place.

Additionally, in some embodiments, the head attaches to the joint using a friction fit. Therefore, a user can attach the head by firmly pressing the attachment stem into the joint and can remove the head by firmly pulling on the head while holding the handle in place.

Other embodiments use one or more magnets to attach the head to the joint. These magnets can be placed on the bottom of the head, the top of the joint or on both. Therefore, because of these magnets, the head will snap or jump into place when it is placed near the joint. Furthermore, when magnets are used, the head will come off with a firm pull.

Furthermore, some embodiments use a head 105 that has a threaded shaft (not shown) to attach to the joint. Thus, to attach or detach the head, the user either screws the head in or out of the joint 125. Moreover, some embodiments use a combination of two or more fastening methods (e.g., a friction fit and snap fit).

### Joint

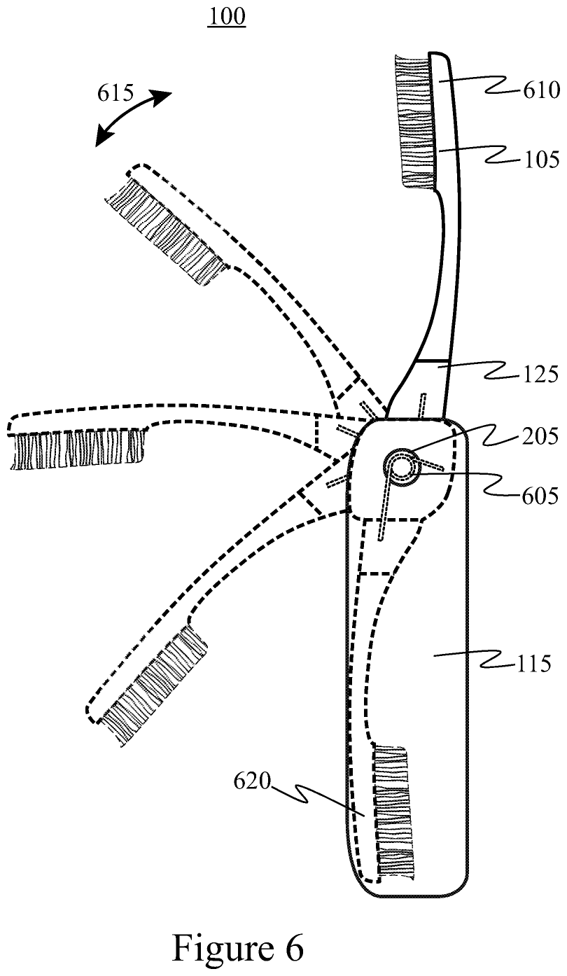

FIG. 6 illustrates a left view of a folding self-cleaning toothbrush. The figure shows the toothbrush transitioning from the first position 610 to the second position 620. The arrow 615 indicates the head's 105 path of motion. In some embodiments, a rotating joint 125 allows the head 105 to rotate into the head storage cavity (i.e., second position 620).

In some embodiments, the joint 125 is between the head 105 and the handle 115. Additionally, the joint 125 allows the head 105 to rotate from the first position 610 with the bristles outside of the handle 115 to the second position 620 with the head folded towards the handle 115 and in which the bristles are contained inside of the recess of the handle. In other words, the rotating joint 125 allows a user to fold the toothbrush from the unfolded position into the folded position and vice versa.

In some embodiments, the toothbrush 100, more specifically the joint 125, is spring-loaded with a spring 605. One end of the spring 605 moves when the joint 125 and head 105 are rotated while the other end of the spring 605 is fixed. In some embodiments, the head is biased by the spring 605, such that the button 205, when pressed with the head in the first position, unlocks the locking mechanism which enables the head 105 to be automatically biased by the spring 605 to move from the second position 620 to the first position 610. Similarly, some embodiments are spring-loaded so that the toothbrush 100 automatically folds when the button 205 is pressed. In other words, in some embodiments the head is biased by the spring to move the head from the first position 610 to the second position 620 when the button 205 is pressed. In some embodiments, the button 205 automatically folds and unfolds the toothbrush 100.

The toothbrush 100 can also use magnets or motors to automatically open and/or close when the button 205 is pressed. For example, a button can signal a motor to rotate the joint to either the folded or unfolded position.

To prevent the toothbrush from rotating while in use (i.e., while a user is brushing his or her teeth), the head 105 locks into position when it reaches either the first position 610 or the second position 620. In other words, some embodiments of the toothbrush comprise of a locking mechanism for locking the head 105 into the first position 610 and/or second position 620.

In some embodiments, a user must push a button 205 to unlock the position of the head 105 in relation to the handle 115. The button 205 disengages the rotation lock and allows the joint 125 to rotate freely. In other words, the button 205 is operable, when pressed, to unlock the locking mechanism for removing the head 105 from the second position 620 and into the first position 610, or vice versa. In some embodiments, the button 205 disengages a mechanical lock. However, in other embodiments, the button is an electronic switch that sends a signal to unlock the rotation of the joint 125.

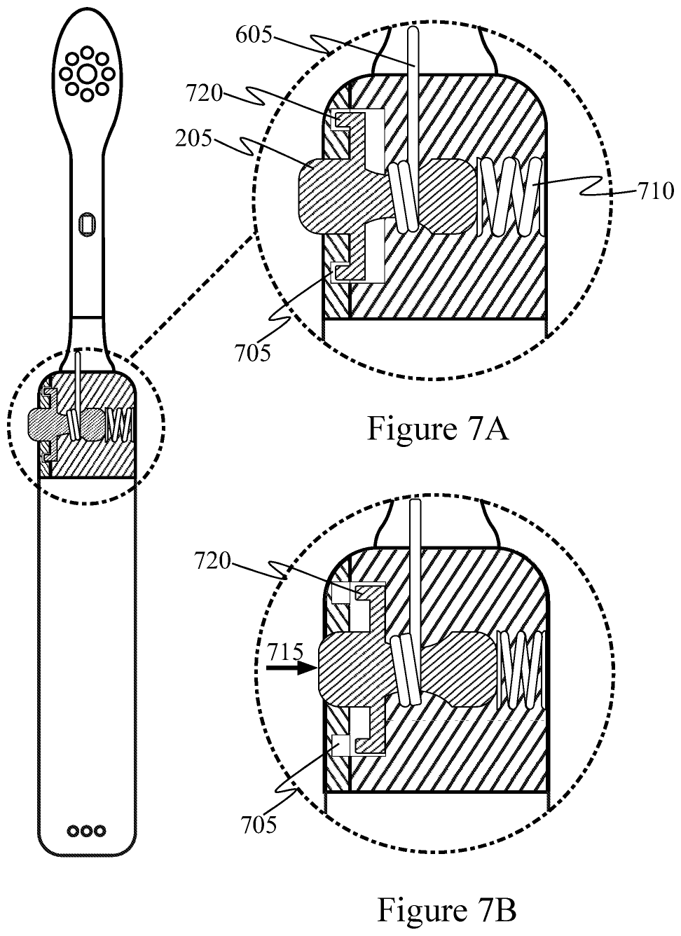

FIG. 7 illustrates one embodiment of a mechanical locking mechanism. The locking mechanism comprises of a button 205 that has arms with protrusions 720. FIG. 7A illustrates the locking mechanism in the engaged position. More particularly, the protrusions 720 of the button 205 are positioned inside the locking cavities 705 of the toothbrush's wall. Thus, the joint and head are prevented from rotating by the locking mechanism. In other words, if a user attempted to rotate the head while the locking mechanism was engaged, the protrusions 720 would collide with the walls of the locking cavities 705 and prevent the rotation.

FIG. 7B illustrates the locking mechanism in the disengaged position. In other words, 7B illustrates the locking mechanism when a force 715 has been applied to the button 205. The force 715 compresses a spring 710 positioned at one end of the button 205 and slides the protrusions 720 out of the locking cavities 705. Once the protrusions 720 leave the locking cavities 705, the joint and head can be rotated. It should be noted that the protrusions 720 will be aligned with the locking cavities 705 when the toothbrush is either in the first position or second position.

In some embodiments, the self-cleaning toothbrush has a sliding joint instead of a rotating joint. Therefore, to store the head, a user can push the unlock button and slide the head into the head storage cavity. Similarly, a user can “unfold” the toothbrush by pushing the unlock button and sliding the head out of the storage chamber.

Overall, the joint allows the head to be protected by the head storage cavity so that the bristles are protected from dust and germs. Additionally, when the toothbrush is in the folded position, the height of the toothbrush is reduced by almost half. Thus, a user can carry the toothbrush in a bag or pocket easily and without having to worry about the bristles contacting contaminated surfaces.

### Handle

FIG. 8 illustrates a perspective view of one embodiment of the self-cleaning toothbrush 800 in the second (i.e., folded) position. The height of the folded toothbrush 800 may vary to accommodate different needs and preferences, from 7 cm to 8 cm, 9 cm, 10 cm, 11 cm, 12 cm, 13 cm or more than 13 cm. Likewise, the width (i.e., from the left side of the handle to the right side of the handle) of the folded toothbrush may vary to accommodate different needs and preferences, from 1 cm to 2 cm, 3 cm, 4 cm or more than 4 cm. Additionally, the depth (i.e., from the back of the handle to the front of the handle) of the folded toothbrush may vary to accommodate different needs and preferences, from 1 cm to 2 cm, 3 cm, 4 cm or more than 4 cm.

In some embodiments, the handle 115 has a slip resistant coating or texture. The slip resistant coating or texture reduces the chances that a user will drop the toothbrush 800.

When the toothbrush 800 is in the second position, the head 105 resides inside of the head storage cavity. In some embodiments, 3 sides of the head 105 are protected by the handle 115. In other words, in the second position, only the back of the head 105 is exposed. In some embodiments, the head 105 creates a water resistant and dust resistant seal with the handle 115 when in the second position. In other words, water and dust cannot enter the head storage cavity or reach the bristles when the toothbrush is in the second position.

The handle 115 may also have one or more storage compartments 805. For example, the handle 115 can have storage compartments on each side of the handle 115. In some embodiments, the storage compartment 805 can be opened by pulling on a tab. Moreover, the storage compartment 805 can be used to store replacement heads, floss, toothpaste and/or medicine.

In some embodiments, the handle 115 also houses a battery. FIG. 9 illustrates a front view and back view of the folded toothbrush 900 and shows the battery 905 in hidden lines. The battery 905 resides below the surface of the right side of the handle. In some embodiments, the battery is in a different location. For example, in some embodiments, the battery is inside the left side of the handle 115 or inside the head 105. Since the battery 905 is enveloped by the handle 115, the battery 905 is protected from dust, water and impact damage.

Since the battery 905 is embedded in the handle 115, the battery 905 is permanent. In other words, a user cannot remove or replace the battery 905. The permanent battery 905 allows the folding self-cleaning toothbrush 900 to have a more streamline design and reduces the number of entry points for water and dust. However, in some embodiments, the toothbrush uses a replaceable battery. For example, in some embodiments, the battery can be accessed, removed and replaced by a user. In some embodiments, the toothbrush has more than one battery.

In some embodiments, the battery 905 is rechargeable via charging contacts 215 on the handle 115. In an alternate embodiment, the charging contacts are on the head. Additionally, in some embodiments, the battery recharges through conventional connecters (e.g., USB, USB-C, Micro USB, Mini USB or Thunderbolt). Some embodiments do not have charging ports or charging connectors. Instead, the embodiment uses inductive charging (i.e., wireless charging). In other words, the toothbrush has an inductive charging unit that is electrically connected to the battery, such that the battery is chargeable through inductive charging. Thus, the toothbrush begins to charge automatically when place on top or near a wireless charging pad or station. Additionally, in some embodiments the handle has solar panels (not shown) which charge the battery. Thus, a user can maintain the battery charged while away from power sources (e.g., while camping).

In some embodiments, the handle 115 has controls to choose the sanitation cycle and strength. For example, the handle 115 can have one or more control buttons 910. In some embodiments, the control buttons 910 are mechanical buttons. In other embodiments, the toothbrush uses capacitive touch buttons. In other words, some embodiments use buttons which do not require an applied force, but instead only require contact with the surface of the control buttons. Additionally, some embodiments allow the user to control the toothbrush through voice commands. For example, some embodiments allow a user to turn on the sanitization device by saying “clean my toothbrush” while near the toothbrush.

In some embodiments, the battery 905 powers the sanitization device, indicator LEDs, motors, and any of the computing systems and transmitting units of the toothbrush 900. For example, the toothbrush of FIG. 9 includes a computing system 915 and a Bluetooth/Wi-Fi transceiver 920. Additionally, in some embodiments, the computer system 915 and transceiver 920 are housed inside the handle 115 and are protected from dust and water by the handle 115.

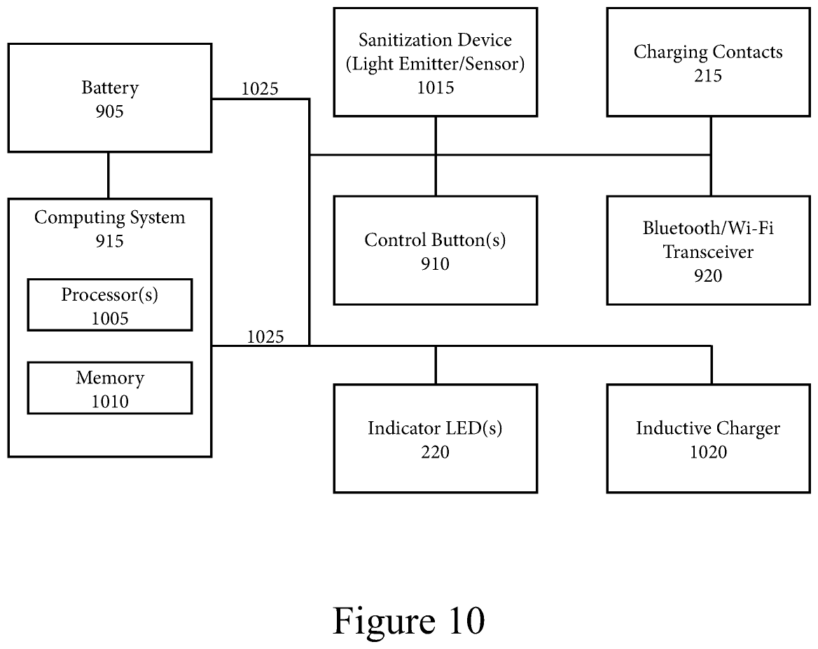

FIG. 10 illustrates the computing system 915 and the electrical wires 1025 that connect all the electronic components of the toothbrush. For example, the computing system 915 is connected to the indicator LEDs 220, battery 905, control buttons 910 (which may include an internal switch in the joint or handle (not shown) for automatically activating the sanitization device), transceiver 920, sanitization device 1015 and inductive charger 1020 through electrical wires 1025. The electrical wires 1025 allow both electricity and data to be transferred between all the electrical components of the toothbrush.

Additionally, the computing system 915 includes a processor 1005 and stored computer-executable instructions in the memory 1010 that are executable by the processor 1005 for generating one or more alerts. For example, the alert can be an alert for replacing the head, an alert for indicating the sanitization device has completed a sanitation cycle, or an alert when the battery is running low. In some embodiments, the user can be notified of the alert by a flashing LED, a noise (i.e., a beep) and/or by a text message sent to their phone or computer.

In some embodiments, the computing system 915 can detect whether there is moisture in the charging ports or on the charging contacts. Thus, the computing system 915 can disable charging to prevent a short circuit if there is liquid in the ports or on the contacts. The computing system 915 can also flash a specific color and pattern on the indicator LED to let a user know that an error is occurring.

Additionally, in some embodiments, the indicator LED can alert a user when the battery 905 is running low. For example, the indicator LED will start blinking red when there is less than 30 minutes of battery life remaining. The LED can also indicate when a user has spent a preset amount of time brushing his or her teeth (e.g., red LED for less than one minute, green LED for two minutes or more). The LED can also indicate when a sanitization cycle is currently underway or has been recently completed.

In some embodiments, the computing system 915 sends and receives commands from the sanitization device 1015, control buttons 910, indicator LEDs 220 and charging contacts 215. Additionally, in some embodiments, the computing system 915 also sends and receives commands to the motors that control the bristles and/or joint rotation. The computing system 915 also receives, stores and analyzes state and use data of the toothbrush.

In some embodiments, the transceiver 920 allows the computing system 915 to connect and transmit data to a cellphone or computer application. Thus, users can select and schedule the sanitization cycles through their phones or computers. Additionally, the Bluetooth and Wi-Fi transceiver 920 can send data to applications on a user's phone or computer which can be used to track and improve oral hygiene habits (e.g., length of time spent brushing and frequency of brushing). In some embodiments, the toothbrush has a Bluetooth transceiver and a separate Wi-Fi adapter.

In some embodiments, the transceiver 920, in conjunction with a user's phone, can be used to track the location of the toothbrush. Thus, the user can use the phone or computer application to find a misplaced toothbrush.

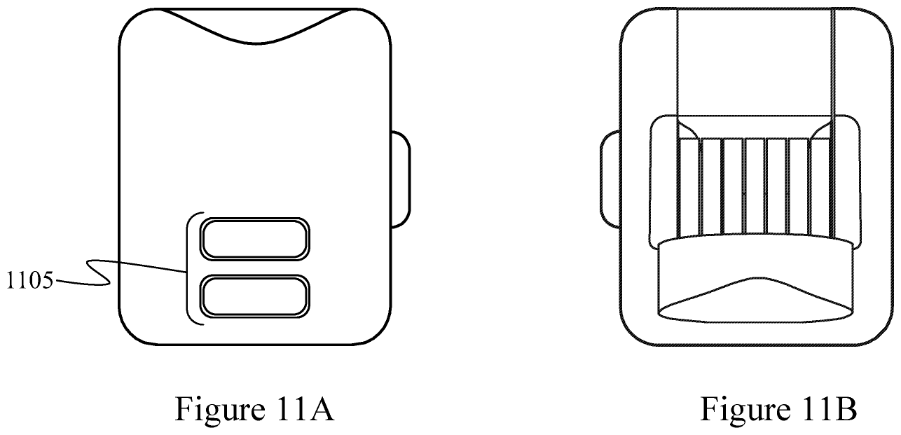

FIG. 11 illustrates a bottom view and top view of one embodiment of the toothbrush that has vents 1105. In some embodiments, the handle has two small vents 1105 on the bottom. However, the vents 1105 can also be placed anywhere along the head storage cavity. Additionally, the vents 1105 can be placed on one or more sides of the handle. In some embodiments, the handle only has one vent. In other embodiments, the handle has many vents (i.e., up to three or more).

One purpose of the vents 1105 is to allow trapped moisture to escape. Reducing the moisture level in the head storage cavity makes it more difficult for germs to thrive and extends the life of the bristles. In some embodiments, the vents 1105 have a mesh screen that protect the head and bristles from debris and dust while still allowing the moisture to escape.

### Sanitization Device

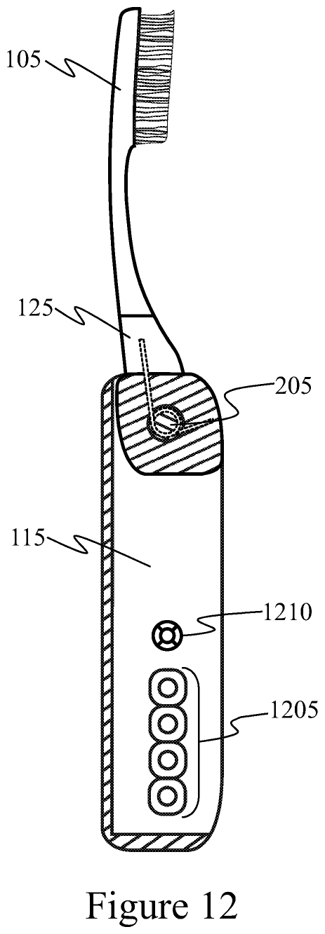

FIG. 12 illustrates a right view, partly in section, of one embodiment of the toothbrush that shows the sanitization device. The sanitization device is operably powered by the battery for emitting a sanitizing light into the recess of the handle when the head 105 is in the second position. Additionally, the light comprises a spectrum of light that is operable to disinfect the bristles.

For example, in some embodiments, the sanitization device consists of one or more UV LEDs 1205 and a sensor 1210. In some embodiments, the UV LEDs 1205 and the sensor 1210 are housed on the inside of the handle. In other words, the LEDs 1205 and sensor 1210 face towards the inside of the head storage cavity. Thus, when the toothbrush is in the second position, the UV LEDs 1205 shine directly on the bristles of the toothbrush. Additionally, the UV LEDs 1205 also eliminate germs on the front and sides of the head 105.

In some embodiments, the sanitizing light consists of a UV-C light (i.e., ultraviolet germicidal irradiation light). The UV-C light emits light with a wavelength between 100 nanometers (nm) to 350 nm. In some embodiments, the sanitization device's emitter emits UV light which is outside the UV-C range (i.e., anywhere between 10 nm to 500 nm or anywhere between 5 nm to 1000 nm). Additionally, in some embodiments the UV-C light is a spectrum of light within the wavelengths of about 230 nm and about 270 nm. Furthermore, the sanitization device kills more than 99% of germs and reduces the moisture content inside the head storage cavity.

In some embodiments, the sanitization device uses UV-C LEDs to emit the sanitizing light. In the illustrated embodiment (see FIG. 12), the sanitization device has 4 UV-C LEDs 1205 in a line array. However, the number of LEDs may vary to accommodate different needs and preferences, from 1 LED to 2 LEDs, 4 LEDs, 5 LEDs, 10 LEDs, 20 LEDs, 40 LEDs, 80 LEDs, 100 LEDs or more than 100 LEDs. In some embodiments, the sanitization device has over 500 LEDs. Additionally, in some embodiments, the LEDs are aligned in a grid. However, in other embodiments, the LEDs are aligned in a circular pattern.

Additionally, some embodiments use organic light-emitting diodes (OLEDs), high-output LEDs (HO-LEDs) or incandescent bulbs. In some embodiments, the sanitization device uses miniature LEDs, miniature OLEDs, miniature high-output LEDs (HO-LEDs) or miniature incandescent bulbs. Some embodiments use a combination of lights for the sanitization device. For example, some sanitization devices use both LEDs and OLEDs. It should be noted that the sanitization device can use any type of light and any number of LEDs.

In some embodiments, the sanitization device has a variety of different modes. For example, the sanitization device can have deep clean mode, overnight mode, quick clean mode, reduce moisture mode, and battery saver mode. Each mode has a preset cycle duration and strength setting. For example, deep clean mode has the highest strength setting (i.e., the brightest light setting) and the longest duration. In contrast, battery saver mode has the lowest strength setting and shortest duration. Thus, a user could use the battery saver mode if he or she was going to be away from a power source for several days or weeks (e.g., while camping or traveling).

Additionally, a user can choose a deep clean mode if the toothbrush has not been sanitized for a few days. Similarly, the user can choose a light cycle if he or she wants to extend the life of the bristles. The sanitization device is also programed to turn off when the preset sanitization cycle is complete.

Additionally, different modes may emit light at different spectrums, intensities and/or for different periods of time. In some embodiments, the preset modes can be modified by the user. More specifically, the control buttons allow the user to control the sanitization device. In other words, the toothbrush has control buttons that are operable to control the sanitization device. For example, the user can extend or shorten a preset mode by either using the control button or by connecting to the toothbrush wirelessly or through a dock.

The sanitization device can also have “smart modes.” For example, the sanitization device can have an automatic mode that stops the sanitization cycle when it detects that the toothbrush is clean. Additionally, in some embodiments, the toothbrush has a safety mechanism that disables the sanitization device if the toothbrush is not properly folded. Thus, a user will not be exposed to the UV light.

In some embodiments, the sanitization device automatically turns on when the sensor 1210 detects that the toothbrush has been folded. For example, a sanitization device will automatically initiate the normal sanitization cycle when the toothbrush has been folded and will turn off after the cycle's preset amount of time. In other words, the sanitization device is activated to emit the sanitizing light for a predetermined amount of time after the folding toothbrush is folded into the second position while the bristles are contained within the recess of the handle.

In some embodiments, the sanitization device turns on when the toothbrush has been docked. Additionally, a user can manually start or stop a sanitization cycle. In some embodiments, the user can also set a daily or weekly time for a cycle to start (e.g., 5 PM every day or Tuesday mornings).

In some embodiments, the folding self-cleaning toothbrush will select a cycle based on usage data. In other words, the toothbrush can analyze a user's data to automatically set times to run the sanitization device. For example, the toothbrush can record the average time of day that the user brushes his or her teeth and start the cycle an hour before that time. Similarly, in some embodiments, the sanitization device can turn on when some predetermine criteria is met. For example, the sanitization device can turn on automatically when it detects too much moisture in the head storage cavity.

Additionally, in some embodiments, the toothbrush has a switch or button (not shown) to disable the sanitization device. Thus, users can disable the sanitization device if they want to save battery or if they are traveling and are worried that the toothbrush will accidentally turn on.

### Dock and Wireless Capabilities

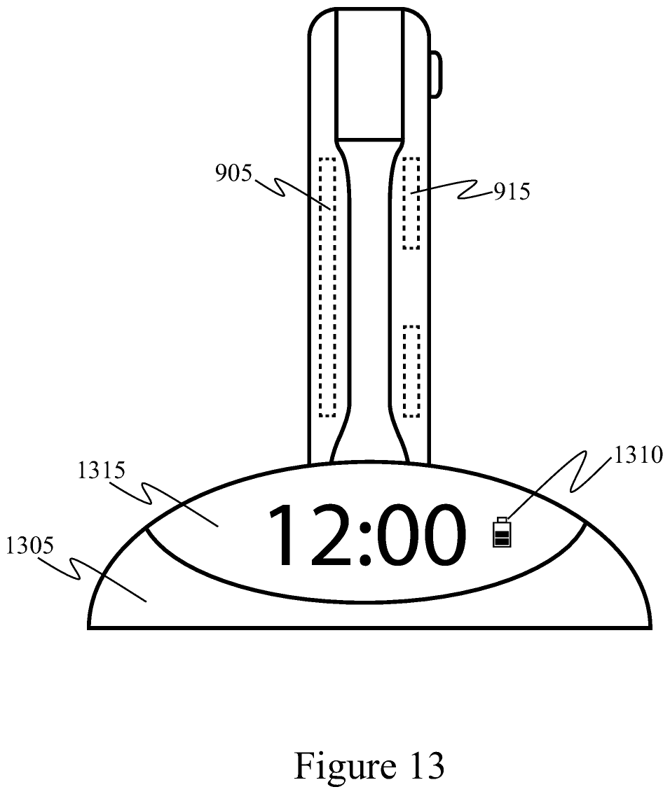

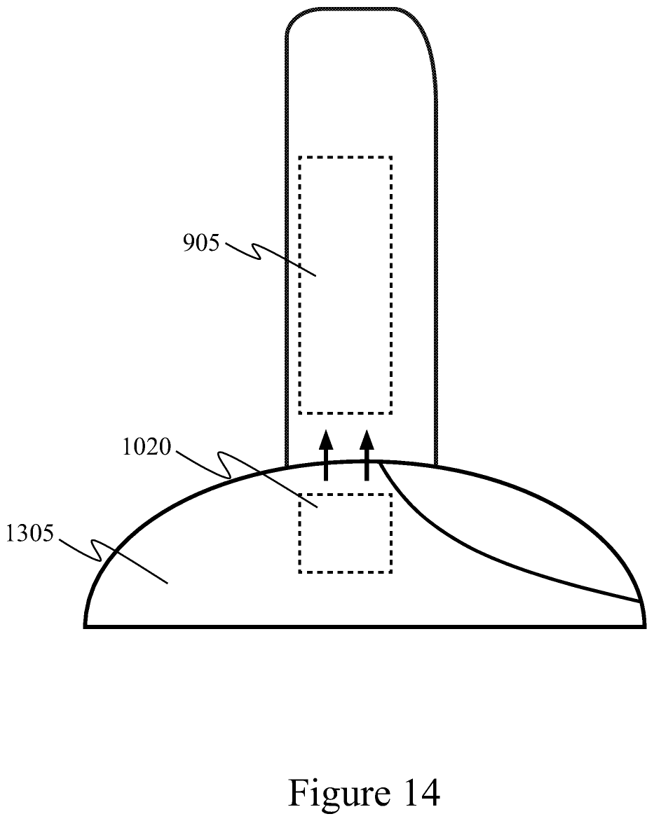

FIGS. 13 and 14 illustrate a front and right view of a toothbrush and a dock 1305 according to one or more embodiments of the present disclosure. In some embodiments, the toothbrush sits vertically in the middle of the dock 1305. The dock 1305 holds the toothbrush in place and charges the folding self-cleaning toothbrush.

In some embodiments, the dock includes a display 1315. For example, in FIG. 13 the display is showing the time. In some embodiments, the dock 1305 includes a charge indicator 1310 which displays the battery's 905 charge level. Additionally, the dock's display 1315 can indicate when it is time to replace the toothbrush head.

In some embodiments, the dock 1305 charges the toothbrush through the charging contacts. Additionally, some embodiments of the dock charge the toothbrush through a charging port or through inductive charging. For example, FIG. 14 illustrates the inductive charger 1020 that is aligned with and connected to the battery 905. In some embodiments, the dock can hold and charge multiple folding self-cleaning toothbrushes.

The dock 1305 can also connect to the toothbrush through the charging mechanism. In other words, the dock can receive and send commands to the computing system 915 of the toothbrush. Similarly, the dock 1305 can transfer data to and from the toothbrush. For example, the dock 1305 can download usage time and battery information from the toothbrush's computing system 915.

In some embodiments, the toothbrush automatically folds when it is placed in a dock 1305. Similarly, in some embodiments, the toothbrush automatically unfolds when it is taken off the dock. In other embodiments, the toothbrush folds when it is placed in the dock and unfolds when it is taken off.

In some embodiments, the dock 1305 allows the sanitization device to use hi-power modes. The hi-power sanitization modes take advantage of the extra energy provided by the dock to clean the toothbrush more thoroughly. Thus, the sanitization device can disinfect the head without draining the battery 905. Additionally, in some embodiments, the sanitization device automatically turns on when the folding toothbrush is connected to the dock.

Overall, the folding self-cleaning toothbrush improves the user's overall experience by providing a disinfected toothbrush head and by allowing them to access the oral hygiene information produced by the folding self-cleaning toothbrush.

### Computer Systems of the Present Disclosure

It will be appreciated that computer systems are increasingly taking a wide variety of forms. In this description and in the claims, the term “computer system” or “computing system” is defined broadly as including any device or system—or combination thereof—that includes at least one physical and tangible processor and a physical and tangible memory capable of having thereon computer-executable instructions that may be executed by a processor. By way of example, not limitation, the term “computer system” or “computing system,” as used herein is intended to include personal computers, desktop computers, laptop computers, tablets, hand-held devices (e.g., mobile telephones, PDAs, pagers), microprocessor-based or programmable consumer electronics, minicomputers, mainframe computers, multi-processor systems, network PCs, distributed computing systems, datacenters, message processors, routers, switches, and even devices that conventionally have not been considered a computing system, such as wearables (e.g., glasses).

The memory may take any form and may depend on the nature and form of the computing system. The memory can be physical system memory, which includes volatile memory, non-volatile memory, or some combination of the two. The term “memory” may also be used herein to refer to non-volatile mass storage such as physical storage media.

The computing system also has thereon multiple structures often referred to as an “executable component.” For instance, the memory of a computing system can include an executable component. The term “executable component” is the name for a structure that is well understood to one of ordinary skill in the art in the field of computing as being a structure that can be software, hardware, or a combination thereof.

For instance, when implemented in software, one of ordinary skill in the art would understand that the structure of an executable component may include software objects, routines, methods, and so forth, that may be executed by one or more processors on the computing system, whether such an executable component exists in the heap of a computing system, or whether the executable component exists on computer-readable storage media. The structure of the executable component exists on a computer-readable medium in such a form that it is operable, when executed by one or more processors of the computing system, to cause the computing system to perform one or more functions, such as the functions and methods described herein. Such a structure may be computer-readable directly by a processor—as is the case if the executable component were binary. Alternatively, the structure may be structured to be interpretable and/or compiled—whether in a single stage or in multiple stages—so as to generate such binary that is directly interpretable by a processor.

The term “executable component” is also well understood by one of ordinary skill as including structures that are implemented exclusively or near-exclusively in hardware logic components, such as within a field programmable gate array (FPGA), an application specific integrated circuit (ASIC), Program-specific Standard Products (ASSPs), System-on-a-chip systems (SOCs), Complex Programmable Logic Devices (CPLDs), or any other specialized circuit. Accordingly, the term “executable component” is a term for a structure that is well understood by those of ordinary skill in the art of computing, whether implemented in software, hardware, or a combination thereof.

The terms “component,” “service,” “engine,” “module,” “control,” “generator,” or the like may also be used in this description. As used in this description and in this case, these terms—whether expressed with or without a modifying clause—are also intended to be synonymous with the term “executable component” and thus also have a structure that is well understood by those of ordinary skill in the art of computing.

While not all computing systems require a user interface, in some embodiments a computing system includes a user interface for use in communicating information from/to a user. The user interface may include output mechanisms as well as input mechanisms. The principles described herein are not limited to the precise output mechanisms or input mechanisms as such will depend on the nature of the device. However, output mechanisms might include, for instance, speakers, displays, tactile output, projections, holograms, and so forth. Examples of input mechanisms might include, for instance, microphones, touchscreens, projections, holograms, cameras, keyboards, stylus, mouse, or other pointer input, sensors of any type, and so forth.

Accordingly, embodiments described herein may comprise or utilize a special purpose or general-purpose computing system. Embodiments described herein also include physical and other computer-readable media for carrying or storing computer-executable instructions and/or data structures. Such computer-readable media can be any available media that can be accessed by a general purpose or special purpose computing system. Computer-readable media that store computer-executable instructions are physical storage media. Computer-readable media that carry computer-executable instructions are transmission media. Thus, by way of example—not limitation—embodiments disclosed or envisioned herein can comprise at least two distinctly different kinds of computer-readable media: storage media and transmission media.

Computer-readable storage media include RAM, ROM, EEPROM, solid state drives (“SSDs”), flash memory, phase-change memory (“PCM”), CD-ROM or other optical disk storage, magnetic disk storage or other magnetic storage devices, or any other physical and tangible storage medium that can be used to store desired program code in the form of computer-executable instructions or data structures and that can be accessed and executed by a general purpose or special purpose computing system to implement the disclosed functionality of the invention. For example, computer-executable instructions may be embodied on one or more computer-readable storage media to form a computer program product.

Transmission media can include a network and/or data links that can be used to carry desired program code in the form of computer-executable instructions or data structures and that can be accessed and executed by a general purpose or special purpose computing system. Combinations of the above should also be included within the scope of computer-readable media.

Further, upon reaching various computing system components, program code in the form of computer-executable instructions or data structures can be transferred automatically from transmission media to storage media (or vice versa). For example, computer-executable instructions or data structures received over a network or data link can be buffered in RAM within a network interface module (e.g., a “NIC”) and then eventually transferred to computing system RAM and/or to less volatile storage media at a computing system. Thus, it should be understood that storage media can be included in computing system components that also—or even primarily—utilize transmission media.

Those skilled in the art will further appreciate that a computing system may also contain communication channels that allow the computing system to communicate with other computing systems over, for example, a network. Accordingly, the methods described herein may be practiced in network computing environments with many types of computing systems and computing system configurations. The disclosed methods may also be practiced in distributed system environments where local and/or remote computing systems, which are linked through a network (either by hardwired data links, wireless data links, or by a combination of hardwired and wireless data links), both perform tasks. In a distributed system environment, the processing, memory, and/or storage capability may be distributed as well.

Those skilled in the art will also appreciate that the disclosed methods may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

Although the subject matter described herein is provided in language specific to structural features and/or methodological acts, it is to be understood that the subject matter defined in the appended claims is not necessarily limited to the described features or acts so described. Rather, the described features and acts are disclosed as example forms of implementing the claims.

### Definitions

Unless defined otherwise, all technical and scientific terms used herein have the same meaning as commonly understood by one of ordinary skill in the art to which the present disclosure pertains.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” as well as variants thereof (e.g., “includes,” “has,” “involves,” “contains,” etc.), and similar terms as used herein, including within the claims, shall be inclusive and/or open-ended, shall have the same meaning as the word “comprising” and variants thereof (e.g., “comprise” and “comprises”), and do not exclude additional un-recited elements or method steps, illustratively.

It will be noted that, as used in this specification and the appended claims, the singular forms “a,” “an” and “the” include plural referents unless the context clearly dictates otherwise. Thus, for example, reference to a singular referent (e.g., “widget”) includes one, two, or more referents. Similarly, reference to a plurality of referents should be interpreted as comprising a single referent and/or a plurality of referents unless the content and/or context clearly dictate otherwise. For example, reference to referents in the plural form (e.g., “widgets”) does not necessarily require a plurality of such referents. Instead, it will be appreciated that independent of the inferred number of referents, one or more referents are contemplated herein unless stated otherwise.

As used herein, directional terms, such as “top,” “bottom,” “left,” “right,” “up,” “down,” “upper,” “lower,” “proximal,” “distal” and the like are used herein solely to indicate relative directions and are not otherwise intended to limit the scope of the disclosure and/or claimed invention.

To facilitate understanding, like reference numerals (i.e., like numbering of components and/or elements) have been used, where possible, to designate like elements common to the figures. Specifically, in the exemplary embodiments illustrated in the figures, like structures, or structures with like functions, will be provided with similar reference designations, where possible. Specific language will be used herein to describe the exemplary embodiments. Nevertheless, it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

Various aspects of the present disclosure can be illustrated by describing components that are bound, coupled, attached, connected, and/or joined together. As used herein, the terms “bound,” “coupled”, “attached”, “connected,” and/or “joined” are used to indicate either a direct association between two components or, where appropriate, an indirect association with one another through intervening or intermediate components. In contrast, when a component is referred to as being “directly bound,” “directly coupled”, “directly attached”, “directly connected,” and/or “directly joined” to another component, no intervening elements are present or contemplated. Furthermore, binding, coupling, attaching, connecting, and/or joining can comprise mechanical and/or chemical association.

### Conclusion

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatuses disclosed herein may be made without departing from the scope of the disclosure or of the invention. Thus, while various aspects and embodiments have been disclosed herein, other aspects and embodiments are contemplated. All changes that come within the meaning and range of equivalency of the claims are to be embraced within their scope.

What is claimed is:

1. A folding self-cleaning toothbrush comprising:
a head with bristles;
a handle, the handle forming a recess that is sized and shaped to receive at least the bristles of the head;
a joint between the head and the handle about which the head can rotate from a first position with the bristles outside of the handle to a second position with the head folded towards the handle and in which the bristles are contained inside of the recess of the handle;
a battery; and
a sanitization device that is operably powered by the battery for emitting a sanitizing light into the recess of the handle when the head is in the second position, the light comprising a spectrum of light that is operable to disinfect the bristles.

2. The folding toothbrush of claim 1, wherein the head comprises a detachable head that is detachable from the handle.

3. The folding toothbrush of claim 1, further comprising of a motor that vibrates the bristles on the head.

4. The folding toothbrush of claim 1, wherein the folding toothbrush comprises a processor and stored computer-executable instructions that are executable by the processor for generating one or more alerts, the one or more alerts comprising one or more of an alert for replacing the head, an alert for indicating the sanitization device has completed a sanitation cycle, or an alert when the battery is running low.

5. The folding toothbrush of claim 1, wherein the sanitizing light consists of a UV-C light.

6. The folding toothbrush of claim 5, wherein the UV-C light is a spectrum of light within the wavelengths of about 230 nm and about 270 nm.

7. The folding toothbrush of claim 5, further comprising control buttons that are operable to control the sanitization device.

8. The folding toothbrush of claim 5, wherein the sanitization device is activated to emit the sanitizing light for a predetermined amount of time after the folding toothbrush is folded into the second position while the bristles are contained within the recess of the handle.

9. The folding toothbrush of claim 1, further comprising a locking mechanism for locking the head into the second position.

10. The folding toothbrush of claim 9, further comprising a button which is operable, when pressed, to unlock the locking mechanism for removing the head from the second position and into the first position.

11. The folding toothbrush of claim 10, wherein the joint is spring-loaded with a spring, such that the button, when pressed with the head in the first position, unlocks the locking mechanism which enables the head to be automatically biased by the spring to move from the second position to the first position.

12. The folding toothbrush of claim 1, further comprising an inductive charging unit that is electrically connected to the battery, such that the battery is chargeable through inductive charging.

13. The folding toothbrush of claim 1, wherein the sanitization device uses UV-C LEDs to emit the sanitizing light.

14. The folding toothbrush of claim 1, further comprising of a Bluetooth transceiver.
